(19) United States
(12) Patent Application Publication
(10) Pub. No.: US 2026/0000806 A1
(43) Pub. Date: Jan. 1, 2026

(54) HYDROCOLLOID-BASED WOUND DRESSINGS AND METHODS FOR THE SAME

(71) Applicant: Neenah, Inc., Alpharetta, GA (US)

(72) Inventors: Pranabes Pramanik, Orangeburg, NY (US); Chau Chen, Greeenfield, MA (US)

(21) Appl. No.: 19/248,874

(22) Filed: Jun. 25, 2025

Related U.S. Application Data
(60) Provisional application No. 63/665,483, filed on Jun. 28, 2024.

(51) Int. Cl. A61L 15/60; A61L 15/20; A61L 15/22; A61L 15/44; C08G 63/00; C08G 71/04

(52) U.S. Cl. CPC A61L 15/60; A61L 15/20; A61L 15/225; A61L 15/44; C08G 63/00; C08G 71/04

(57) ABSTRACT

Hydrocolloid-based wound dressings and backing layers thereof are described. The backing layer may include a carrier film prepared from a carrier film composition. The carrier film composition may include a surface active agent and a combination of first and second aromatic polyester-based thermoplastic polyurethanes. The combination of the first and second aromatic polyester-based thermoplastic polyurethanes may provide a wrinkle-free carrier film under accelerated aging conditions. The hydrocolloid-based wound dressing may include an adhesive layer and the backing layer disposed on the adhesive layer. Methods for preparing the backing layer may include contacting the first aromatic polyester-based thermoplastic polyurethane, the second aromatic polyester-based thermoplastic polyurethane, and the surface active agent with one another to prepare a mixture. The method may also include melting and homogenizing the mixture in an extruder, and extrusion casting the melted mixture on the carrier substrate to prepare the backing layer.

### CROSS-REFERENCE TO RELATED APPLICATIONS

This application claims the benefit of U.S. Provisional Application No. 63/665,483, filed Jun. 28, 2024, the complete disclosure of which is incorporated herein by reference for all purposes.

### TECHNICAL FIELD

This following description generally relates to hydrocolloid-based wound dressings and carrier films or backing layers thereof.

### BACKGROUND

Hydrocolloid-based wound dressings are often utilized to facilitate wound healing and prevent infections of wounds. Conventional hydrocolloid-based wound dressings may often include a backing layer, which may include a carrier film or layer disposed on a carrier substrate, a hydrocolloid layer prepared from water-soluble or water-swellable hydrocolloids, and a release liner. The hydrocolloids may be capable of or configured to absorb water, swell, and liquify to form a gel over the wound to thereby enhance or facilitate wound healing and prevent infections. While conventional hydrocolloid-based wound dressings have proven to be effective, improved hydrocolloid-based wound dressings and backing layers thereof would provide further improved stability and/or shelf life of the hydrocolloid-based wound dressings.

### BRIEF SUMMARY

This following is intended merely to introduce a simplified summary of some aspects of one or more implementations of the subject matter discussed herein. Further areas of applicability of the subject matter will become apparent from the detailed description provided hereinafter. This summary is not an extensive overview, nor is it intended to identify key or critical elements of the present teachings, nor to delineate the scope of the subject matter. Rather, its purpose is merely to present one or more concepts in simplified form as a prelude to the detailed description below.

The foregoing and/or other aspects and utilities described herein may be achieved by providing a backing layer for a hydrocolloid-based wound dressing. The backing layer may include a carrier film prepared from a carrier film composition. The carrier film composition may include a surface active agent and a combination of a first aromatic polyester-based thermoplastic polyurethane and a second aromatic polyester-based thermoplastic polyurethane. The surface active agent may include a silicone.

In one aspect, the backing layer may further include a carrier substrate. The carrier film may be disposed on the carrier substrate.

In one aspect, the weight ratio of the first aromatic polyester-based thermoplastic polyurethane to the second aromatic polyester-based thermoplastic polyurethane may be from about 1.5:1 to about 2.6:1.

In one aspect, the first aromatic polyester-based thermoplastic polyurethane may be present in an amount of from about 45 wt % to about 80 wt %, based on the total weight of the carrier film composition.

In one aspect, the second aromatic polyester-based thermoplastic polyurethane may be present in an amount of from about 20 wt % to about 40 wt %, based on the total weight of the carrier film composition.

In one aspect, the first aromatic polyester-based thermoplastic polyurethane may have a Shore Hardness, as determined according to reference test ASTM D2240, of from about 80A to about 70D.

In one aspect, the first aromatic polyester-based thermoplastic polyurethane may have a specific gravity, as determined according to reference test ASTM D792, of from about 0.9 to about 1.5.

In one aspect, the first aromatic polyester-based thermoplastic polyurethane may have a tensile strength, as determined according to reference test ASTM D412/D638, of from about 40 MPa to about 70 MPa.

In one aspect, the first aromatic polyester-based thermoplastic polyurethane may have a tensile stress at about 50% elongation, according to reference test ASTM D412/D638, of from about 6 MPa to about 12 MPa.

In one aspect, the first aromatic polyester-based thermoplastic polyurethane may have a tensile stress at about 100% elongation, according to reference test ASTM D412/D638, of from about 10 MPa to about 14 MPa.

In one aspect, the first aromatic polyester-based thermoplastic polyurethane may have an elongation at break, according to reference test ISO 527-3/5/500 or reference test ASTM D412/D638, of from about 260% to about 320%.

In one aspect, the first aromatic polyester-based thermoplastic polyurethane may have a tear strength, according to reference test ISO 34-1B, of from about 65 kN/m to about 85 kN/m.

In one aspect, wherein the first aromatic polyester-based thermoplastic polyurethane may have a Kofler Melting Point of from about 65° C. to about 95° C.

In one aspect, the first aromatic polyester-based thermoplastic polyurethane may have a viscosity at about 23° C. of from about 100 centipoise (cP) to about 200 cP.

In one aspect, the first aromatic polyester-based thermoplastic polyurethane may have: a peel strength adhesion to polyethylene terephthalate (PET), according to reference test TP-141, of from about 0.19 kN/m to about 0.25 KN/m; a peel strength to aluminum foil, according to reference test ASTM D1867-72, of from about 0.8 kN/m to about 1.0 kN/m; and/or a peel strength to mylar film, according to reference test ASTM D1867-72, of from about 4 kN/m to about 6 kN/m.

In one aspect, the first aromatic polyester-based thermoplastic polyurethane may have a Vicat Softening Point, according to reference test ASTM D1525, of from about 40° C. to about 70° C.

In one aspect, the first aromatic polyester-based thermoplastic polyurethane may have a glass transition temperature (Tg), as determined with a differential scanning calorimeter (DSC), of from about 15° C. to about 30° C.

In one aspect, the second aromatic polyester-based thermoplastic polyurethane may have a Shore Hardness, as determined according to reference test ASTM D2240, of from about 70 A to about 100 A.

In one aspect, the second aromatic polyester-based thermoplastic polyurethane may have a specific gravity, as determined according to reference test ASTM D792, of from about 0.9 to about 1.5.

In one aspect, the second aromatic polyester-based thermoplastic polyurethane may have a tensile strength, as determined according to reference test ASTM D412/D638, of from about 30 MPa to about 70 MPa.

In one aspect, the second aromatic polyester-based thermoplastic polyurethane may have an elongation at break, according to reference test ISO 527-3/5/500 or reference test ASTM D412/D638, of from about 350% to about 600%.

In one aspect, the second aromatic polyester-based thermoplastic polyurethane may have a 100% modulus at 100% strain, according to reference test ASTM D412/D638 of the ASTM, of from about 3.0 MPa to about 7 MPa.

In one aspect, the second aromatic polyester-based thermoplastic polyurethane may have a 300% modulus at 300% strain, according to reference test ASTM D412/D638 of the ASTM, of from about 10 MPa to about 16 MPa.

In one aspect, the second aromatic polyester-based thermoplastic polyurethane may have a melt viscosity at about 204° C., according to reference test ASTM D1084-88, of from about 24,000 cP to about 32,000 cP.

In one aspect, the second aromatic polyester-based thermoplastic polyurethane may have: a peel strength adhesion to polyethylene terephthalate (PET), according to reference test TP-141, of from about 0.09 kN/m to about 0.13 kN/m; a peel strength to aluminum foil, according to reference test ASTM D1867-72, of from about 0.8 kN/m to about 1.0 kN/m; and/or a peel strength to mylar film, according to reference test ASTM D1867-72, of from about 4 kN/m to about 6 kN/m.

In one aspect, the second aromatic polyester-based thermoplastic polyurethane may have a glass transition temperature (Tg), as determined with a differential scanning calorimeter (DSC), of from about 15° C. to about 30° C.

In one aspect, the second aromatic polyester-based thermoplastic polyurethane may have a ring and ball softening point, as determined according to ASTM E2892, of from about 150° C. to about 170° C.

In one aspect, the surface active agent may further include one or more of a siloxane, polytetrafluoroethylene (PTFE), an antistatic agent, or a combination thereof.

In one aspect, the surface active agent may include the siloxane dispersed in an organic resin.

In one aspect, the siloxane may include a polydimethylsiloxane. The organic resin may include a thermoplastic polyurethane resin.

In one aspect, the siloxane may be present in an amount of from greater than 0 wt % to about 8 wt %, based on the total weight of the carrier film composition.

In one aspect, the carrier film composition may include an additive.

In one aspect, the additive may include diatomaceous earth.

In one aspect, the diatomaceous earth may have an average particle diameter of from about 1 μm to about 10 μm.

In one aspect, the backing layer may have a moisture vapor transmission rate, according to reference test ASTM E96-00, of from about 450 g/m²/day to about 600 g/m²/day.

In one aspect, the backing layer may have an elongation at break of from about 400% to about 850%.

In one aspect, a release force between the carrier film and the carrier substrate is from greater than 0 N to about 1 N.

The foregoing and/or other aspects and utilities described herein may be achieved by providing a hydrocolloid-based wound dressing. The hydrocolloid-based wound dressing may include any one of the backing layers described herein, and an adhesive layer disposed on the backing layer.

In one aspect, the adhesive layer may include a hydrocolloid and a tackifier.

In one aspect, the hydrocolloid-based wound dressing may further include a release liner disposed on the adhesive layer.

In one aspect, the hydrocolloid-based wound dressing may have a moisture vapor transmission rate, according to reference test ASTM E96-00, of from about 100 g/m²/day to about 180 g/m²/day.

In one aspect, the hydrocolloid-based wound dressing may have a modulus at 100% elongation of from about 3.5 N to about 5.5 N.

In one aspect, the hydrocolloid-based wound dressing may have an elongation at break of from about 550% to about 700%.

In one aspect, a release force between the release liner and the adhesive layer may be from about 10 N to about 16 N.

The foregoing and/or other aspects and utilities described herein may be achieved by providing a method for preparing any one of the backing layers described herein. The method may include contacting the first aromatic polyester-based thermoplastic polyurethane, the second aromatic polyester-based thermoplastic polyurethane, and the surface active agent with one another to prepare a mixture. The method may also include melting and homogenizing the mixture in an extruder. The method may further include extrusion casting the melted mixture on the carrier substrate to prepare the backing layer.

In one aspect, the method may further include drying the mixture prior to melting and homogenizing the mixture in the extruder.

In one aspect, the mixture may be substantially free of organic solvents.

In one aspect, the method may further include annealing the backing layer after extrusion casting the melted mixture on the carrier substrate.

Further areas of applicability of the subject matter will become apparent from the detailed description provided hereinafter. It should be understood that the detailed description and specific examples, while indicating some typical aspects of the subject matter, are intended for purposes of illustration only and are not intended to limit the scope thereof.

The recitation herein of desirable objects which may be met by various embodiments of the present description is not meant to imply or suggest that any or all of these objects may be present as essential features, either individually or collectively, in the most general embodiment of the present description or any of its more specific embodiments.

### BRIEF DESCRIPTION OF THE DRAWINGS

The accompanying drawings, which are incorporated in and constitute a part of this specification, illustrate several embodiments of the subject matter and, together with the description, serve to explain the principles thereof.

FIG. 1 illustrates a schematic cross-sectional view of an exemplary hydrocolloid-based wound dressing, according to one or more implementations discussed herein.

### DETAILED DESCRIPTION

This description and the accompanying drawings illustrate exemplary embodiments and should not be taken as limiting, with the claims defining the scope of the present description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Except as otherwise noted, any quantitative values are approximate whether the word “about” or “approximately” or the like are stated or not. The materials, methods, and examples described herein are illustrative only and not intended to be limiting.

As used throughout, ranges are used as shorthand for describing each and every value that is within the range. It should be appreciated and understood that the description in a range format is merely for convenience and brevity and should not be construed as an inflexible limitation on the scope of any embodiments or implementations discussed herein. Accordingly, the range should be construed to have specifically included all the possible subranges as well as individual numerical values within that range. As such, any value within the range may be selected as the terminus of the range. For example, description of a range such as from 1 to 5 should be considered to have specifically included subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Additionally, all numerical values are “about” or “approximately” the indicated value, and take into account experimental error and variations that would be expected by a person having ordinary skill in the art. It should be appreciated that all numerical values and ranges discussed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is discussed herein, any numerical value falling within the range is also specifically included.

As used herein, the expression “free” of a material of a substance may refer to a composition, component, or phase where the material is present in an amount of less than 1.0 wt %, less than 0.1 wt %, less than 0.05 wt %, less than 0.01 wt %, less than 0.005 wt %, or less than 0.0001 wt %, based on a total weight of the composition, component, or phase. As used herein, the expression “substantially free” of a material or substance may refer to a composition, component, or phase where the material is present in an amount of about 1.0 wt % or more and less than 20.0 wt %, less than 10.0 wt %, less than 5.0 wt %, less than 3.0 wt %,

All references cited herein are hereby incorporated by reference in their entireties. In the event of a conflict in a definition with a cited reference, the present teachings control.

Wound dressings, particularly, hydrocolloid-based wound dressings or laminates are described. Hydrocolloid-based wound dressings are a type of wound dressing often utilized in the treatment of exuding wounds, such as minor burns, cuts, or the like, to reduce wound pain and/or facilitate wound healing by providing a moist healing environment or by absorbing exudate. The hydrocolloid-based wound dressings are waterproof; thus, permitting washing, bathing or showering while also preventing the spread of pathogenic microorganisms. The hydrocolloid-based wound dressings often include multiple layers, which may include, inter alia, a carrier film, a hydrocolloid adhesive, a release liner, or combinations thereof. Each of the respective layers of the hydrocolloid-based wound dressing may be or include varying components and/or compositions. As such, the respective properties of each of the layers of the hydrocolloid-based wound dressing may exhibit varying changes when stored for an extended period of time, thereby resulting in delamination, shrinking, and/or wrinkling of one or more of the layers thereof. The hydrocolloid-based wound dressings described herein demonstrate improved or enhanced stability, efficacy, and/or performance as compared to conventional hydrocolloid-based wound dressings. Particularly, the present inventors have surprisingly and unexpectedly discovered that hydrocolloid-based wound dressings that utilize carrier film compositions including a combination of a first aromatic polyester-based thermoplastic polyurethane and a second aromatic polyester-based thermoplastic polyurethane, a surface active agent (e.g., polydimethylsiloxane), an additive (e.g., diatomaceous earth), or a combination thereof, exhibited comparable or enhanced stability with respect to wrinkling and shrinking, moisture vapor transmission rate, elongation at break, and modulus, as compared to conventional or comparative hydrocolloid-based wound dressings. Particularly, the present inventors have surprisingly and unexpectedly discovered that hydrocolloid-based wound dressings that utilize carrier film compositions including a combination of a first aromatic polyester-based thermoplastic polyurethane and a second aromatic polyester-based thermoplastic polyurethane, and a surface active agent (e.g., a polydimethylsiloxane dispersion) in the thermoplastic polyurethane, exhibited comparable or enhanced stability with respect to wrinkling and shrinking, moisture vapor transmission rate, elongation at break, and modulus, as compared to conventional or comparative hydrocolloid-based wound dressings.

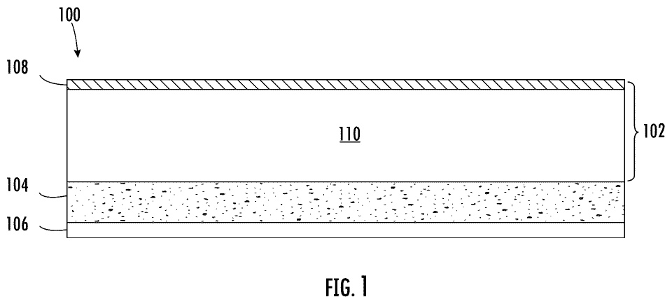

FIG. 1 illustrates a schematic cross-sectional view of an exemplary hydrocolloid-based wound dressing or laminate 100, according to one or more implementations. The hydrocolloid-based wound dressing 100 may include one or more of a backing layer 102, an adhesive layer 104, a release liner 106, or a combination thereof. For example, as illustrated in FIG. 1, the hydrocolloid-based wound dressing 100 may include the backing layer 102, the adhesive layer 104 disposed adjacent the backing layer 102, and the release liner 106 disposed adjacent the adhesive layer 104. While FIG. 1 illustrates the hydrocolloid-based wound dressing 100 as including the backing layer 102, the adhesive layer 104, and the release liner 106, it should be appreciated that the hydrocolloid-based wound dressing 100 may include additional layers (not shown) or a plurality of any one or more of the backing layers 102, the adhesive layer 104, and the release liner 106. For example, the hydrocolloid-based wound dressing 100 may include a wound- release layer (not shown) that may be disposed directly adjacent the adhesive layer 104 and/or interposed between the adhesive layer 104 and the release liner 106. The wound-release layer may be capable of or configured to contact the wound and facilitate removal of the hydrocolloid-based wound dressing 100 from the wound after application. Illustrative wound-release layers may be or include, but are not limited to, a porous film, a net, or the like, or any combination thereof.

The backing layer 102 may be an outermost layer of the hydrocolloid-based wound dressing 100. The backing layer 102 may be flexible, water-resistant or water impermeable, breathable, or a combination thereof. The backing layer 102 may be capable of or configured to protect the wound from external contamination and prevent water or bacteria from contacting the wound. The backing layer 102 may also be capable of or configured to allow for the exchange of air and moisture vapor to facilitate wound healing. The backing layer 102 may include a carrier substrate 108, a carrier layer or film 110 including a carrier film composition, or a combination thereof. For example, as illustrated in FIG. 1, the backing layer 102 may include the carrier substrate 108 and the carrier film 110, prepared from the carrier film composition (e.g., thermoplastic polyurethane composition), disposed on or adjacent the carrier substrate 108. The carrier substrate 108 may be any substrate capable of or configured to support the carrier film 110. For example, the carrier substrate 108 may be a paper-based carrier substrate, a polymer-based carrier substrate, a thermoplastic-based carrier substrate, or a combination thereof. While FIG. 1 illustrates the backing layer 102 as having the carrier substrate 108 and the carrier film 110, it should be appreciated that the backing layer 102 may only include the carrier film 110. For example, the carrier substrate 108 (e.g., paper-based carrier substrate) may be utilized in the manufacture of the backing layer 102 and subsequently removed, leaving only the carrier film 110. Accordingly, the carrier substrate 108 may be utilized to facilitate the manufacture of the backing layer (e.g., via melt extrusion casting), but may be excluded from the backing layer 102 of the hydrocolloid-based wound dressing 100.

The backing layer 102 may have any suitable thickness. For example, the thickness of the backing layer 102 may be from about 0.01 mm to about 0.1 mm, about 0.01 to about 0.05, about 0.02 mm to about 0.04 mm, or about 0.03 mm. In another example, the thickness of the backing layer 102 may be from about 0.01 mm, about 0.02 mm, or about 0.03 mm to about 0.04 mm, about 0.05 mm, about 0.06 mm, about 0.07 mm, about 0.08 mm, about 0.09 mm, or about 0.1 mm.

The carrier film 110 of the backing layer 102 may have a thickness of from about 0.01 mm to about 0.1 mm, about 0.01 to about 0.05, about 0.02 mm to about 0.04 mm, or about 0.03 mm. The carrier film 110 of the backing layer 102 may also have a thickness of from about 0.01 mm, about 0.02 mm, or about 0.03 mm to about 0.04 mm, about 0.05 mm, about 0.06 mm, about 0.07 mm, about 0.08 mm, about 0.09 mm, or about 0.1 mm.

It should be appreciated that the thickness of the hydrocolloid-based wound dressing 100 and/or one or more components thereof may be determined according to ASTM D6988-21 and/or via a digital micrometer. For example, the thickness of the hydrocolloid-based wound dressing 100, the backing layer 102, and/or the carrier film 110 may be determined according to ASTM D6988-21.

The carrier film composition of the carrier film 110 may be capable of or configured to provide improved stability, efficacy, and/or performance of one or more properties (e.g., elongation, modulus, moisture vapor transmission rate, etc.) of the hydrocolloid-based wound dressing 100 as compared to conventional hydrocolloid-based wound dressings. The carrier film composition of the carrier film 110 may include one or more of a thermoplastic, a surface active agent, an additive, or any combination thereof.

The thermoplastic of the carrier film composition may be or include one or more thermoplastic polymers. For example, the thermoplastic may be or include a single thermoplastic polymer. In another example, the thermoplastic may be or include a plurality of thermoplastic polymers. The plurality of thermoplastic polymers may be a homogenous or heterogenous mixture or blend thereof. The plurality of thermoplastic polymers may be a single-phase blend or a multi-phase blend. A single-phase blend of thermoplastic polymers may include a plurality of thermoplastic polymers that are miscible with one another and/or form a polymeric material, often with a single glass transition temperature (Tg) based on the ratio of the thermoplastic polymers. A multi-phase blend may include two or more thermoplastic polymers that are immiscible with one another and/or have a plurality of glass transition (Tg) temperatures. As used herein, the term or expression “thermoplastic polymer” may refer to a plastic capable of being repeatedly softened or melted by increases in temperature and hardened by decreases in temperature. The one or more thermoplastic polymers may be crystalline or semi-crystalline. A crystalline thermoplastic polymer may generally include no crosslinking to a modest amount of crosslinking relative to a thermoset polymer. The relative decreased amount of crosslinking has a correspondingly reduced influence on respective properties of the thermoplastic polymer, such as softening and/or melting. A thermoplastic polymer may also generally include a higher molecular weight polymer relative to a thermoset, as a relatively higher molecular weight may enhance one or more properties, such as melting point to enhance or facilitate the integrity of the thermoplastic during use.

In an exemplary implementation, the thermoplastic polymer may be or include one or more thermoplastic elastomers. An elastomer may include a macromolecular material that may return rapidly to approximately the initial dimensions and/or shape after deformation by stress or a biasing force. The thermoplastic-based elastomer may be or include any one or more suitable thermoplastic elastomers. For example, the thermoplastic-based elastomer may be or include a single thermoplastic elastomer. In another example, the thermoplastic-based elastomer may be or include a plurality (e.g., two or more) of thermoplastic elastomers. The plurality of thermoplastic elastomers may be a homogenous or heterogeneous mixture or blend. The thermoplastic elastomer may have crosslinks capable of or configured to prevent permanent deformation during use via increased elasticity and/or decreased plasticity. The one or more thermoplastic elastomers may have one or more crosslinks about 4,000 to about 10,000 monomer units in the respective polymeric chain.

In an exemplary implementation, the thermoplastic elastomer includes one or more thermoplastic polyurethane elastomers. For example, the thermoplastic elastomer may be or include a blend of two or more thermoplastic polyurethane elastomers. The thermoplastic polyurethane elastomer may include a block copolymer including a hard segment and a soft segment. The hard segment may include a polyisocyanate (e.g., diisocyanate), a chain extender, or a combination thereof. The chain extender may be or include, but is not limited to, one or more of a polyol (e.g., a diol, a glycol, etc.), a polyfunctional amine (e.g., a primary amine, a secondary amine, or the like, or a combination thereof), or the like, or a combination thereof. For example, the chain extender may be or include, but is not limited to, one or more of a 1,4-butanediol, a glycol, a diol, an ethylene glycol, a diamine, or the like, or a combination thereof. The soft segment may include a long chain diol, a long chain elastomeric polyol, or a combination thereof. Illustrative long chain diols and long chain elastomeric polyols may be or include, but are not limited to, one or more of a polyether, a polyether polyol, a polycaprolactone polyester, a polyadipate polyester, a polytetramethylene glycol ether, a hydroxyl group end-capped monomer, a hydroxyl group end-capped, a polyester, a polyester polyol, or the like, or a combination thereof. Illustrative polyether polyols may be or include, but are not limited to, a diol and/or a triol having a molecular weight of from about 4,000 Da to about 6,000 Da or more. Illustrative polyesters may be or include, but are not limited, to a polyester prepared from a glycol, such as an ethylene glycol, and an adipic acid having a molecular weight of from about 1,000 Da to about 3,000 Da and/or a poly(epsilon-caprolactone). Illustrative polyesters may also be or include, but are not limited, to a polyester prepared from a polycarboxylic acid, such as a dicarboxylic acid (e.g., an adipic acid) and a polyol. The polyester may have a hydroxy moiety at one or more termini. In an exemplary implementation, the thermoplastic polyurethane elastomer may be a reaction product or prepared from reacting one or more diisocyanates with one or more long chain diols and/or the chain extender. The thermoplastic polyurethane elastomer may include one or more crosslinks.

It should be appreciated that the type of the soft segment polyol utilized to prepare the thermoplastic polyurethane elastomer may at least partially determine or facilitate the classification thereof. For example, the soft segment polyol of the thermoplastic polyurethane elastomer may classify the thermoplastic polyurethane elastomer as a polyether thermoplastic polyurethane elastomer or polyether-based thermoplastic polyurethane, a polyester thermoplastic polyurethane elastomer or polyester-based thermoplastic polyurethane, a polycaprolactam-based polyurethane, or the like. Particularly, a polyether polyol and a polyester polyol may be utilized as the polyol of the soft segment to prepare a polyether-based thermoplastic polyurethane and a polyester-based thermoplastic polyurethane, respectively. A polyester-based thermoplastic polyurethane may include, but is not limited to, a hydroxy terminated polyester polyol having a molecular weight of up to about 2,500 Da, about 3,000 Da, about 3,500 Da, about 4,000 Da, or more. The polyester-based thermoplastic polyurethane may be prepared by step growth and/or condensation polymerization, as is conventionally known in the art. The polyether polyol of a polyether-based thermoplastic polyurethane may be prepared using an epoxide by addition, ring opening, and/or anionic polymerization. The “hard segment” of the thermoplastic polyurethane elastomer may include a “chain extender” polyol, which may include a short chain diol, such as, a 1,4-butanediol, a 1,6-hexanediol, and/or an ethylene glycol, and a diisocyanate, such as, a 4,4′-diphenylmethane diisocyanate (“MDI”), a hydrogenated 4,4′-diphenylmethane diisocyanate (“HMDI”), a hexamethylene diisocyanate (“HDI”), a toluene diisocyanate (“TDI”), and/or a 1,5-diisocyanate, or the like, or any combination thereof. The hard segment may be capable of or configured to hydrogen bonding with a different chain to promote or facilitate crystallization. Illustrative thermoplastic polyurethanes may be or include, but are not limited to, one or more of an aromatic polycaprolactam polyurethane, an aliphatic polycaprolactam polyurethane, an aromatic polyester-based thermoplastic polyurethane, a linear polyester-based thermoplastic polyurethane, an aliphatic polyester-based polyurethane, an aliphatic polyether-based polyurethane, an aromatic polyether-based polyurethane, or a combination thereof.

The thermoplastic(s) may include one or more additives. For example, the thermoplastic, the thermoplastic elastomer, and/or the thermoplastic polyurethane elastomer may include one or more additives. Illustrative additives may be or include, but are not limited to, one or more of a catalyst, a catalyst neutralizer, a chain transfer agent capable of or configured to control termination of a polymer chain and polymerization of another polymer chain, a filler (e.g., carbon black, barite, clay, chalk, calcium carbonate, titanium dioxide, or the like, or any combination thereof), a reinforcing agent, a plasticizer, a softener and/or processing aid, an antioxidant, a curing agent, a surfactant, an accelerator, a fire retardant, a colorant, a retarder, a resin, a fatty acid and/or fatty acid soap, a bonding agent, or the like, or any combination thereof.

In an exemplary implementation, the thermoplastics of the carrier film includes a plurality of polyester-based thermoplastic polyurethane elastomers. The polyester-based thermoplastic polyurethane elastomers may be or include crystalline and/or semi-crystalline polymers. In an exemplary implementation, the plurality of polyester-based thermoplastic polyurethane elastomers includes one or more of an aromatic polyester-based thermoplastic polyurethane, a linear polyester-based thermoplastic polyurethane, an aliphatic polyester-based polyurethane, or a combination thereof. Illustrative polyester-based thermoplastic polyurethanes may be or include, but are not limited to, those commercially available from Lubrizol Corporation of Wickliffe, Ohio under the general designation ESTANE® and/or PEARLSTICK™. For example, the polyester-based thermoplastic polyurethane may be or include one or more of the following: ESTANE® 5701, which is a polyester-based polyurethane polymer; ESTANE® 5701 F1, which is an aromatic polyester-based polyurethane polymer; ESTANE® 5702, which is an aromatic polyester-based polyurethane polymer; ESTANE® 5703, which is an aromatic polyester based polyurethane polymer; ESTANE® 5707 F1, which is an aromatic polyester-based polyurethane polymer; ESTANE® 5712 F30, which is an aromatic polyester-based polyurethane polymer; ESTANE® 5713 F2, which is an aromatic polyester-based polyurethane; ESTANE® 58213 NAT, which is a polyester based thermoplastic polyurethane; PEARLSTICK® 5715, which is an aromatic polyester-based thermoplastic polyurethane, PEARLSTICK® 5701, which is a polyester-based thermoplastic polyurethane, or the like, or any combination thereof.

In an exemplary implementation, the plurality of polyester-based thermoplastic polyurethane elastomers includes, consists essentially of, or consists of a first polyester-based thermoplastic polyurethane elastomer and a second polyester-based thermoplastic polyurethane elastomer. The first and second polyester-based thermoplastic polyurethanes may be or include aromatic polyester-based thermoplastic polyurethanes. For example, the plurality of polyester-based thermoplastic polyurethane elastomers may include a combination of a first aromatic polyester-based thermoplastic polyurethane and a second aromatic polyester-based thermoplastic polyurethane. Each of the first and second aromatic polyester-based thermoplastic polyurethane polymers or elastomers may have a molecular weight of from about 1,000 Da to about 250,000 Da, about 1,000 Da to about 200,000 Da, about 1,000 Da to about 150,000 Da, or about 1,000 Da to about 100,000 Da.

The first aromatic polyester-based thermoplastic polyurethane may have a Shore Hardness of from about 80 A to about 70 D. For example, the first aromatic polyester-based thermoplastic polyurethane may have a Shore Hardness of from about 80 A, about 85 A, about 90 A, or about 95 A to about 100 A, about 60 D, about 65 D, or about 70 D. In another example, the first aromatic polyester-based thermoplastic polyurethane may have a Shore Hardness of from about 80 A to about 70 D, about 85 A to about 65 D, about 90 A to about 60 D, about 90 A to about 100 A, or about 95 A. The Shore Hardness may be evaluated according to reference test ASTM D2240 of the American Society for Testing and Materials (ASTM) or reference test ISO 868 of the International Organization for Standardization (ISO). For example, the Shore Hardness may be evaluated according to ASTM D2240-15 (2021).

The first aromatic polyester-based thermoplastic polyurethane may have a specific gravity of from about 0.9 to about 1.5. For example, the first aromatic polyester-based thermoplastic polyurethane may have a specific gravity of from about 0.9, about 1.0, about 1.1, or about 1.2 to about 1.3, about 1.4, or about 1.5. In another example, the first aromatic polyester-based thermoplastic polyurethane may have a specific gravity of from about 0.9 to about 1.4 g/cm³, about 1.1 to about 1.3, or about 1.2. The specific gravity may be evaluated or determined according to reference test ISO 2781 of the ISO or reference test ASTM D792 of the ASTM. For example, the specific gravity may be evaluated or determined according to method A of reference test ASTM D792-20.

In at least one implementation, the first aromatic polyester-based thermoplastic polyurethane may have a tensile strength, as determined according to reference test ASTM D412/D638 of the ASTM or reference test ISO 527-3/5/500 of the ISO, of from about 40 MPa to about 70 MPa. For example, the first aromatic polyester-based thermoplastic polyurethane may have a tensile strength of from about 40 MPa, about 45 MPa, about 50 MPa, or about 53 MPa to about 57 MPa, about 60 MPa, about 65 MPa, or about 70 MPa. In another example, the first aromatic polyester-based thermoplastic polyurethane may have a tensile strength of from about 40 MPa to about 70 MPa, about 45 MPa to about 65 MPa, about 50 MPa to about 60 MPa, about 52 MPa to about 57 MPa, or about 54 MPa. In another implementation, the first aromatic polyester-based thermoplastic polyurethane may have a tensile strength, as determined according to reference test ISO 527-3/5A/200 of the ISO, of from about 30 to about 50 MPa. For example, the first aromatic polyester-based thermoplastic polyurethane may have a tensile strength of from about 30 MPa to about 50 MPa, about 35 MPa to about 45 MPa, about 37 MPa to about 42 MPa, or about 39 MPa.

The first aromatic polyester-based thermoplastic polyurethane may have a tensile stress at 50% elongation of from about 6 MPa to about 12 MPa. For example, the first aromatic polyester-based thermoplastic polyurethane may have a tensile stress at 50% elongation of from about 6 MPa, about 6.5 MPa, about 7 MPa, about 7.5 MPa, about 8 MPa, or about 8.5 MPa to about 9 MPa, about 9.5 MPa, about 10 MPa, about 10.5 MPa, about 11 MPa, about 11.5 MPa, or about 12 MPa. In another example, the first aromatic polyester-based thermoplastic polyurethane may have a tensile stress at 50% elongation of from about 6 MPa to about 12 MPa, about 6.5 MPa to about 11.5 MPa, about 7 MPa to about 11 MPa, about 7.5 MPa to about 10.5 MPa, about 8 MPa to about 10 MPa, about 8.5 MPa to about 9.5 MPa, or about 8.7 MPa. The tensile stress may be evaluated according to reference test ISO 527-3/5/500 of the ISO.

The first aromatic polyester-based thermoplastic polyurethane may have a tensile stress at 100% elongation of from about 10 MPa to about 14 MPa. For example, the tensile stress of the first aromatic polyester-based thermoplastic polyurethane at 100% elongation may be from about 10 MPa, about 10.5 MPa, about 11 MPa, or about 11.5 MPa to about 12 MPa, about 12.5 MPa, about 13 MPa, or about 13.5 MPa. In another example, the tensile stress at 100% elongation may be from about 10 MPa to about 13.5 MPa, about 10.5 MPa to about 13 MPa, about 11 MPa to about 12.5 MPa, about 11.5 MPa to about 12 MPa, or about 11.7 MPa.

The first aromatic polyester-based thermoplastic polyurethane may have an elongation at break of from about 260% to about 320%. For example, the elongation at break of the first aromatic polyester-based thermoplastic polyurethane may be from about 260%, about 270%, about 280%, or about 285% to about 295%, about 300%, about 310%, or about 320%. In another example, the elongation at break of the first aromatic polyester-based thermoplastic polyurethane may be from about 260% to about 320%, about 270% to about 310%, about 280% to about 300%, about 285% to about 295%, or about 290%. The elongation at break may be evaluated according to reference test ISO 527-3/5/500 of the ISO.

The first aromatic polyester-based thermoplastic polyurethane may have a tear strength (B) of from about 65 kN/m to about 85 kN/m. For example, the first aromatic polyester-based thermoplastic polyurethane may have a tear strength (B) of from about 65 kN/m to about 85 kN/m, about 70 kN/m to about 80 kN/m, about 73 kN/m to about 77 kN/m, or about 75 kN/m. The tear strength (B) may be evaluated according to reference test ISO 34-1B of the ISO.

The first aromatic polyester-based thermoplastic polyurethane may have a Kofler Melting Point of from about 65° C. to about 95° C. For example, the first aromatic polyester-based thermoplastic polyurethane may have a Kofler Melting Point of from about 65° C. to about 95° C., about 70°° C. to about 90° C., about 75° C. to about 85° C., about 78° C. to about 82° C., or about 80° C.

The first aromatic polyester-based thermoplastic polyurethane may be soluble in one or more solvents. For example, the first aromatic polyester-based thermoplastic polyurethane may be soluble in methylethylketone (MEK), dimethylformamide (DMF), tetrahydrofuran (THF), cyclohexanone, or a combination thereof. The first aromatic polyester-based thermoplastic polyurethane may swell in ethylacetate.

The first aromatic polyester-based thermoplastic polyurethane may have a viscosity of from about 100 mPa·s or centipoise (cP) to about 200 cP. For example, a 20% total solids solution of the first aromatic polyester-based thermoplastic polyurethane in methylethylketone (MEK) may have a viscosity of from about 100 cP to about 200 cP, about 120 cP to about 180 cP, or about 140 cP to about 160 cP. The viscosity may be determined using a Viscometer at a temperature of about 23° C. For example, the viscosity may be determined with a Brookfield Viscometer with a number 2 spindle at about 20 RPM and about 23° C.

The first aromatic polyester-based thermoplastic polyurethane may have a peel strength adhesion to polyethylene terephthalate (PET), according to reference test TP-141, of from about 0.19 kN/m to about 0.25 KN/m or about 0.22 kN/m. The first aromatic polyester-based thermoplastic polyurethane may also have a peel strength to aluminum foil, according to reference test ASTM D1867-72, of from about 0.8 kN/m to about 1.0 kN/m or about 0.9 kN/m. The first aromatic polyester-based thermoplastic polyurethane may also have a peel strength to mylar film, according to reference test ASTM D1867-72, of from about 4 kN/m to about 6 kN/m or about 5 kN/m.

The first aromatic polyester-based thermoplastic polyurethane may have a Vicat Softening Point, according to reference test ASTM D-1525, of from about 40° C. to about 70° C. For example, the first aromatic polyester-based thermoplastic polyurethane may have a Vicat Softening Point of from about 40° C. to about 70° C., about 45° C. to about 65° C., about 50° C. to about 60° C., or about 55° C.

The first aromatic polyester-based thermoplastic polyurethane may have a glass transition temperature (Tg), as determined with a differential scanning calorimeter (DSC), of from about 15° C. to about 30° C. For example, the first aromatic polyester-based thermoplastic polyurethane may have a glass transition temperature of from about 15° C. to about 30° C., about 20° C. to about 25° C., about 22° C. to about 24° C., or about 23° C.

The second aromatic polyester-based thermoplastic polyurethane elastomer may have a Shore Hardness of from about 70 A to about 100 A. For example, the second aromatic polyester-based thermoplastic polyurethane may have a Shore Hardness of from about 70 A, about 75 A, about 80 A, or about 82 A to about 87 A, about 90 D, about 95 D, or about 100 D. In another example, the second aromatic polyester-based thermoplastic polyurethane may have a Shore Hardness of from about 70 A to about 100 A, about 75 A to about 95 A, about 80 A to about 90 D, about 82 A to about 87 A, or about 85 A. The Shore Hardness may be evaluated according to reference test ASTM D2240 of the American Society for Testing and Materials (ASTM) or reference test ISO 868 of the International Organization for Standardization (ISO).

The second aromatic polyester-based thermoplastic polyurethane may have a specific gravity of from about 0.9 to about 1.5. For example, the second aromatic polyester-based thermoplastic polyurethane may have a specific gravity of from about 0.9, about 1.0, about 1.1, or about 1.2 to about 1.3, about 1.4, or about 1.5. In another example, the second aromatic polyester-based thermoplastic polyurethane may have a specific gravity of from about 0.9 to about 1.5, about 1.0 to about 1.4, about 1.1 to about 1.3, or about 1.2. The specific gravity may be evaluated or determined according to reference test ISO 2781 of the ISO or reference test ASTM D792 of the ASTM.

In at least one implementation, the second aromatic polyester-based thermoplastic polyurethane may have a tensile strength of from about 30 MPa to about 70 MPa. For example, the second aromatic polyester-based thermoplastic polyurethane may have a tensile strength of from about 30 MPa, about 35 MPa, about 40 MPa, or about 45 MPa to about 55 MPa, about 60 MPa, about 65 MPa, or about 70 MPa. In another example, the second aromatic polyester-based thermoplastic polyurethane may have a tensile strength of from about 30 MPa to about 70 MPa, about 35 MPa to about 65 MPa, about 40 MPa to about 60 MPa, about 45 MPa to about 55 MPa, about 48 MPa to about 50 MPa, or about 49 MPa. The tensile strength may be determined according to reference test ASTM D412/D638 of the ASTM or reference test ISO 527-3/5/500 of the ISO. In an exemplary implementation, the tensile strength is determined according to reference test ASTM D638.

The second aromatic polyester-based thermoplastic polyurethane may have an elongation at break of from about 350% to about 600%. For example, the elongation at break of the second aromatic polyester-based thermoplastic polyurethane may be from about 350%, about 375%, about 400%, about 425%, or about 450% to about 470%, about 500%, about 525%, about 550%, about 575%, or about 600%. In another example, the elongation at break of the second aromatic polyester-based thermoplastic polyurethane may be from about 350% to about 600%, about 375% to about 575%, about 400% to about 550%, about 425% to about 525%, about 450% to about 470%, or about 460%. The elongation at break may be evaluated according to reference test ISO 527-3/5/500 of the ISO or reference test ASTM D412-16 of the ASTM. In an exemplary implementation, the elongation at break is evaluated according to reference test ASTM D412-16 of the ASTM, and crescent test pieces are utilized.

The second aromatic polyester-based thermoplastic polyurethane may have a 100% modulus at 100% strain, according to reference test ASTM D412/D638 of the ASTM, of from about 3.0 MPa to about 7 MPa. For example, the second aromatic polyester-based thermoplastic polyurethane may have a 100% modulus at 100% strain of from about 3.0 MPa to about 7.0 MPa, about 3.2 MPa to about 6.2 MPa, about 3.5 MPa to about 6.5 MPa, about 4.0 MPa to about 6.0 MPa, about 4.5 MPa to about 5.9 MPa, and about 4.8 MPa to about 5.6 MPa, or about 5.2 MPa. In another example, the second aromatic polyester-based thermoplastic polyurethane may have a 100% modulus at 100% strain of from about 3.0 MPa, about 3.2 MPa, about 3.5 MPa, about 4.0 MPa, about 4.5 MPa, or about 4.8 MPa to about 5.6 MPa, about 5.9 MPa, about 6.0 MPa, about 6.5 MPa, about 6.2 MPa, or about 7.0 MPa.

The second aromatic polyester-based thermoplastic polyurethane may have a 300% modulus at 300% strain, according to reference test ASTM D412/D638 of the ASTM, of from about 10 MPa to about 16 MPa. For example, the second aromatic polyester-based thermoplastic polyurethane may have a 300% modulus at 300% strain of from 10 MPa to about 16 MPa, about 10.5 MPa to about 15.7 MPa, about 11.0 MPa to about 15.5 MPa, about 11.5 MPa to about 15.3 MPa, about 12.0 MPa to about 15.1 MPa, about 12.5 MPa to about 14.9 MPa, about 13.0 MPa to about 14.7 MPa, and about 13.3 MPa to about 14.5 MPa. In another example, the second aromatic polyester-based thermoplastic polyurethane may have a 300% modulus at 300% strain of from about 10 MPa, about 10.5 MPa, about 11.0 MPa, about 11.5 MPa, about 12.0 MPa, about 12.5 MPa, or about 13.0 MPa to about 13.3 MPa, about 14.5 MPa, about 14.7 MPa, about 14.9 MPa, about 15.1 MPa, about 15.3 MPa, about 15.5 MPa, about 15.7 MPa, or about 16 MPa.

The second aromatic polyester-based thermoplastic polyurethane may have a viscosity of from about 400 mPa·s or centipoise (cP) to about 550 cP. For example, a 15% total solids solution of the second aromatic polyester-based thermoplastic polyurethane in tetrahydrofuran (THF) may have a viscosity of from about 400 cP to about 550 cP, about 425 cP to about 525 cP, about 450 cP to about 500 cP, or about 475 cP. The viscosity may be determined using a Viscometer at a temperature of about 23° C. For example, the viscosity may be determined with a Brookfield Viscometer with a number 2 spindle at about 20 RPM and about 23° C.

The second aromatic polyester-based thermoplastic polyurethane may also have a viscosity of from about 2,000 mPa·s or centipoise (cP) to about 2,800 cP. For example, a 15% total solids solution of the second aromatic polyester-based thermoplastic polyurethane in cyclohexanone may have a viscosity of from about 2,000 cP to about 2,800 cP, about 2,100 cP to about 2,700 cP, about 2,200 cP to about 2,600 cP, or about 2,300 cP to about 2,500 cP, or about 2,400 cP. The viscosity may be determined using a Viscometer at a temperature of about 23° C. For example, the viscosity may be determined with a Brookfield Viscometer with a number 2 spindle at about 20 RPM and about 23° C.

The second aromatic polyester-based thermoplastic polyurethane may also have a melt viscosity at about 204° C. (or about 400° F.), according to reference test ASTM D1084/88, of from about 24,000 mPa·s or centipoise (cP) to about 32,000 cP. For example, the second aromatic polyester-based thermoplastic polyurethane in cyclohexanone may have a melt viscosity of from about 24,000 cP to about 32,000 cP, about 25,000 cP to about 31,000 cP, about 26,000 cP to about 30,000 cP, about 27000 cP to about 29,000 cP, or about 28,000 cP.

The second aromatic polyester-based thermoplastic polyurethane may have a peel strength adhesion to polyethylene terephthalate (PET), according to reference test TP-141, of from about 0.09 kN/m to about 0.13 kN/m or about 0.11 kN/m. The second aromatic polyester-based thermoplastic polyurethane may also have a peel strength to aluminum foil, according to reference test ASTM D1867-72, of from about 0.8 kN/m to about 1.0 kN/m or about 0.9 kN/m. The second aromatic polyester-based thermoplastic polyurethane may also have a peel strength to mylar film, according to reference test ASTM D1867-72, of from about 4 kN/m to about 6 kN/m or about 5 kN/m.

The second aromatic polyester-based thermoplastic polyurethane may have a Vicat Softening Point, according to reference test ASTM D-1525, of from about 55° C. to about 85° C. For example, the second aromatic polyester-based thermoplastic polyurethane may have a Vicat Softening Point of from about 55° C. to about 85° C., about 60° C. to about 80° C., about 65° C. to about 75° C., or about 70°° C.

The second aromatic polyester-based thermoplastic polyurethane may have a glass transition temperature (Tg), as determined with a differential scanning calorimeter (DSC), of from about 15° C. to about 30° C. For example, the second aromatic polyester-based thermoplastic polyurethane may have a glass transition temperature of from about 15° C. to about 30° C., about 20° C. to about 25° C., about 22° C. to about 24° C., or about 23° C. In another implementation, the second aromatic polyester-based thermoplastic polyurethane may have a glass transition temperature (Tg) of from about 20° C. to about 36° C. For example, the second aromatic polyester- based thermoplastic polyurethane may have a glass transition temperature (Tg) of from about 22° C. to about 36° C., about 24° C. to about 34° C., about 26° C. to about 32° C., or about 28° C.

The second aromatic polyester-based thermoplastic polyurethane may have a ring and ball softening point, as determined according to ASTM E2892, of from about 150° C. to about 170° C. For example, the second aromatic polyester-based thermoplastic polyurethane may have ring and ball softening point of from about 150° C. to about 170° C., about 152° C. to about 168° C., about 154° C. to about 166° C., about 156° C. to about 164° C., about 158° C. to about 162° C., or about 161° C.

The second aromatic polyester-based thermoplastic polyurethane may have an open time of hot melt adhesive, as determined according to ASTM D4497-94, of less than 10 seconds. For example, the second aromatic polyester-based thermoplastic polyurethane may have an open time of hot melt adhesive of less than 10 seconds, less than 8 seconds, or less than 5 seconds.

The first aromatic polyester-based thermoplastic polyurethane may be present in an amount of from about 45 wt % to about 80 wt %, based on the total weight of the carrier film composition. For example, the first aromatic polyester-based thermoplastic polyurethane may be present in an amount of from about 45 wt %, about 45 wt %, about 50 wt %, about 55 wt %, about 60 wt %, or about 65 wt % to about 70 wt %, about 75 wt %, or about 80 wt %, based on the total weight of the carrier film composition. In another example, the first aromatic polyester-based thermoplastic polyurethane may be present in an amount of from about 50 wt % to about 80 wt %, about 55 wt % to about 75 wt %, about 60 wt % to about 70 wt %, about 65 wt % to about 69 wt %, about 52 wt % to about 67 wt %, or about 67 wt %, based on the total weight of the carrier film composition. In another example, the first aromatic polyester-based thermoplastic polyurethane may be present in an amount of from about 46 wt % to about 58 wt %, about 48 wt % to about 56 wt %, about 50 wt % to about 54 wt %, or about 52 wt %, based on the total weight of the carrier film composition. In yet another example, the first aromatic polyester-based thermoplastic polyurethane may be present in an amount of from about 46 wt %, about 48 wt %, about 50 wt %, or about 51 wt % to about 53 wt %, about 55 wt %, about 60 wt %, or about 65 wt %, based on the total weight of the carrier film composition.

The second aromatic polyester-based thermoplastic polyurethane may be present in an amount of from about 20 wt % to about 40 wt %, based on the total weight of the carrier film composition. For example, the second aromatic polyester-based thermoplastic polyurethane may be present in an amount of from about 20 wt %, about 22 wt %, about 24 wt %, about 26 wt %, or about 28 wt % to about 30 wt %, about 32 wt %, about 34 wt %, about 36 wt %, about 38 wt %, or about 40 wt %, based on the total weight of the carrier film composition. In another example, the second aromatic polyester-based thermoplastic polyurethane may be present in an amount of from about 20 wt % to about 40 wt %, about 22 wt % to about 38 wt %, about 24 wt % to about 36 wt %, about 26 wt % to about 34 wt %, about 28 wt % to about 32 wt %, or about 28 wt % or about 29 wt %, based on the total weight of the carrier film composition.

The first and second aromatic polyester-based thermoplastic polyurethanes may be present in a weight ratio of from about 1.5:1 to about 2.6:1. For example, the weight ratio of the first aromatic polyester-based thermoplastic polyurethane to the second aromatic polyester-based thermoplastic polyurethane may be from about 1.5:1 to about 2.6:1, about 1.6:1 to about 2.5:1, about 1.7:1 to about 2.4:1, about 1.8:1 to about 2.3:1, about 1.9:1 to about 2.2:1, or about 2:1 to about 2.1:1. In another example, the weight ratio of the first aromatic polyester-based thermoplastic polyurethane to the second aromatic polyester-based thermoplastic polyurethane may be from about 2:1 to about 2.6:1, about 2.1:1 to about 2.5:1, about 2.2:1 to about 2.4:1, or about 2.3:1. In yet another example, the weight ratio of the first aromatic polyester-based thermoplastic polyurethane to the second aromatic polyester-based thermoplastic polyurethane may be from about 1.5:1 to about 2.2:1, about 1.6:1 to about 2.1:1, about 1.7:1 to about 2:1, about 1.8:1 to about 1.0:1, or about 1.85:1.

The surface active agent of the carrier film composition may be capable of or configured to facilitate, increase, or otherwise enhance the release of the carrier film 110 from a substrate, such as the carrier substrate 108. The surface active agent may be or include one or more of a silicone, a siloxane, a siloxane masterbatch, polytetrafluoroethylene (PTFE), an antistatic agent, or the like, or any combination thereof. Illustrative surface active agents may be or include, but are not limited to, an ultra-high molecular weight siloxane polymer, an ultra-high molecular weight siloxane polymer solution and/or dispersion, a polydimethylsiloxane, a polydimethylsiloxane solution and/or dispersion, or the like, or any combination thereof. In at least one example, the surface active agent includes an ultra-high molecular weight siloxane polymer dispersion in an organic resin. For example, the surface active agent may include about 50% of polydimethylsiloxane dispersed in a thermoplastic polyurethane resin. Commercially available surface active agents may be or include those in the MB-50 series, such as MB50-017, from MULTIBASE™ from DuPont of Wilmington, DE.

The surface active agent may be present in an amount of from greater than 0 wt % to about 8 wt %, based on the total weight of the carrier film composition. For example, the surface active agent (e.g., polydimethylsiloxane) may be present in an amount of from greater than 0 wt %, about 1 wt %, about 2 wt %, about 3 wt %, or about 3.5 wt % to about 4.5 wt %, about 5 wt %, about 6 wt %, about 7 wt %, or about 8 wt %, based on the total weight of the carrier film composition. In another example, the surface active agent may be present in an amount of from greater than 0 wt % to about 8 wt %, about 1 wt % to about 7 wt %, about 2 wt % to about 6 wt %, about 3 wt % to about 5 wt %, about 3.5 wt % to about 4.5 wt %, or about 4 wt %, based on the total weight of the carrier film composition.

The carrier film composition may include one or more additives. Illustrative additives may be or include, but are not limited to, active agents or actives, wound care agents, hemostatic agents, odor inhibiting substances, diatomaceous earth, or the like, or any combination thereof.

In an exemplary implementation, the one or more additives may be or include diatomaceous earth (DE) capable of or configured to improve or enhance one or more physical properties of the hydrocolloid-based wound dressing 100 or the backing layer 102 thereof. For example, the diatomaceous earth may be capable of or configured to increase the opacity of the backing layer 102. The diatomaceous earth may also be capable of or configured to modify (i.e., increase or decrease) the modulus and/or elongation of the hydrocolloid-based wound dressing 100 or the backing layer 102 thereof. In another example, the diatomaceous earth may be capable of or configured to reduce odors. The diatomaceous earth may be in the form of a powder. For example, the diatomaceous earth may have an average or mean particle diameter of from about 1 μm to about 10 μm. For example, the diatomaceous earth may have an average or mean particle diameter of from about 1 μm to about 10 μm, about 2 μm to about 9 μm, about 3 μm to about 8 μm, about 4 μm to about 7 μm, or about 5 μm. The diatomaceous earth may be in the form of a suspension or a dispersion. The diatomaceous earth may be or include diatomaceous earth particles dispersed in an organic resin, such as a thermoplastic polyurethane resin. For example, the diatomaceous earth may be a dispersion or suspension including about 35 wt % to about 45 wt %, or about 40 wt %, of diatomaceous earth particles in the first and/or second aromatic polyester-based thermoplastic polyurethane. In an exemplary implementation, the diatomaceous earth is utilized as a 40% dispersion in the first aromatic polyester-based thermoplastic polyurethane. The dispersion of the diatomaceous earth may include fumed silica; for example, in an amount of from about 0.1 wt % to about 0.5 wt % or about 0.3 wt %, based on the total weight of the dispersion.

The diatomaceous earth may be present in the carrier film composition in an amount of from greater than 0 wt % to about 30 wt %, based on the total weight of the carrier film composition. For example, the diatomaceous earth may be present in the carrier film composition in an amount of from greater than 0 wt %, about 5 wt %, about 10 wt %, or about 15 wt % to about 20 wt %, about 25 wt %, or about 30 wt %, based on the total weight of the carrier film composition. In another example, the diatomaceous earth may be present in an amount of from greater than 0 wt % to about 30 wt %, about 5 wt % to about 25 wt %, about 10 wt % to about 20 wt %, about 15 wt % or about 16 wt %, based on the total weight of the carrier film composition. In at least one implementation, the carrier layer 110 or the carrier film composition thereof does not include the diatomaceous earth.

The adhesive layer 104 may be an adhesive wound contact layer capable of or configured to directly contact the wound. The adhesive layer 104 may also be capable of or configured to at least partially absorb liquids (e.g., exudate) and/or form a hydrogel to retain the liquid under pressure. The adhesive layer 104 may also be capable of or configured to facilitate healing and/or adhere the hydrocolloid-based wound dressing 100 to the wound. Illustrative adhesive layers 104 may be or include, but are not limited to, hydrocolloids, alginates, carboxymethylcellulose (CMC) fibers, absorbent polymers, hydrocolloid adhesives, hydrogel adhesives, polyurethane adhesives, polyacrylate adhesives, silicones, acrylic adhesives, rubber-based adhesives, latex adhesives, hydrocolloids with tackifiers (e.g., rosin based tackifier), or the like, or any combination thereof. In an exemplary implementation, the adhesive layer 104 includes, a hydrocolloid adhesive, such as a hydrocolloid with a rosin based tackifier.

The adhesive layer 104 may have any suitable thickness. For example, the adhesive layer 104 may have a thickness of from about 0.3 mm to about 0.6 mm, about 0.35 mm to about 0.55 mm, about 0.4 mm to about 0.5 mm, or about 45 mm. In another example, the adhesive layer 104 may have a thickness of from about 0.3 mm, about 0.35 mm, about 0.4 mm, or about 0.45 mm to about 0.50 mm, about 0.55 mm, or about 0.6 mm or greater.

The release liner 106 may be or include any suitable material capable of or configured to be removed before contact between the hydrocolloid-based wound dressing 100 and the wound. Conventional release liners 106 are well known in the art. Illustrative release liners 106 may be or include, but are not limited to, silicone paper release films, silicone paper release strips, paper release liner, any conventional release liner, or the like. The release liner 106 may have a thickness of from about 0.05 mm to about 0.3 mm. For example, the release liner 106 may have a thickness of from about 0.05 mm, about 0.10 mm, or about 0.15 mm to about 0.20 mm, about 0.25 mm, or about 0.3 mm.

In an exemplary implementation, the hydrocolloid-based wound dressing 100 and/or one or more components thereof exhibit comparable or enhanced stability and/or efficacy as compared to conventional hydrocolloid-based wound dressings utilizing alternative compositions for the carrier layer 110 under accelerated aging conditions. Particularly, the hydrocolloid-based wound dressing 100, the backing layer 102, and/or the carrier layer 110 thereof does not exhibit wrinkling (e.g., wrinkle free) after exposure to accelerated aging that mimics at least five (5) years of aging. Additionally, the hydrocolloid-based wound dressing 100, the backing layer 102, and/or the carrier layer 110 exhibits comparable or enhanced performance and/or efficacy with respect to one or more properties as compared to conventional hydrocolloid-based wound dressings utilizing alternative carrier film compositions (e.g., polyether-type TPU, aromatic polyether-type TPU, etc.). The one or more properties may be or include, but are not limited to, modulus at 100% elongation, thickness, release force between the carrier layer 110 and the carrier substrate 108, release force between the release liner 106 and the adhesive layer 104, moisture vapor transmission rate (MVTR), peel adhesion, translucency, flexibility, absorption of liquids (e.g., exudate), adsorption of liquids, or the like, or any combination thereof. The accelerated aging conditions may include exposing the hydrocolloid-based wound dressing 100 to a temperature of about 40° C. at about 30% relative humidity (RH) for at least seven (7) days or more. In an exemplary implementation, the backing layer 102 or the carrier layer 110 thereof exhibits no film wrinkling upon or after exposure to accelerated aging conditions.

The hydrocolloid-based wound dressing 100 may have a moisture vapor transmission rate, at about 37° C., of from about 100 g/m²/day to about 180 g/m²/day. As used herein, the moisture vapor transmission rate (MVTR) measures the degree of breathability of substance, component, material, film, composite, composition, or the like. The MVTR may be evaluated according to reference test ASTM E96-00 or ASTM E96 of the American Society for Testing and Materials (ASTM). The MVTR of the hydrocolloid-based wound dressing 100 may be from about 100 g/m²/day, about 110 g/m²/day, about 120 g/m²/day, about 130 g/m²/day, about 135 g/m²/day, or about 137 g/m²/day to about 141 g/m²/day, about 145 g/m²/day, about 150 g/m²/day, about 155 g/m²/day, about 160 g/m²/day, about 170 g/m²/day, or about 180 g/m²/day. In another example, the MVTR of the hydrocolloid-based wound dressing 100 may be from about 100 g/m²/day to about 180 g/m²/day, about 110 g/m²/day to about 170 g/m²/day, about 120 g/m²/day to about 160 g/m²/day, about 130 g/m²/day to about 150 g/m²/day, about 135 g/m²/day to about 145 g/m²/day, about 137 g/m²/day to about 141 g/m²/day, or about 139 g/m²/day.

The backing layer 102 or the carrier layer 110 may have a MVTR at about 37° C. of from about 450 g/m²/day to about 600 g/m²/day, about 475 g/m²/day to about 575 g/m²/day, about 500 g/m²/day to about 550 g/m²/day, about 525 g/m²/day to about 545 g/m²/day, or about 535 g/m²/day. In another example, the backing layer 102 or the carrier layer 110 thereof may have an MVTR at about 37° C. of from about 450 g/m²/day, about 475 g/m²/day, about 500 g/m²/day, about 525 g/m²/day, or about 530 g/m²/day to about 535 g/m²/day, about 545 g/m²/day, about 550g/m²/day, about 575 g/m²/day, or about 600 g/m²/day.

The hydrocolloid-based wound dressing 100 may have a modulus at 100% elongation of from about 3.5 N to about 5.5 N. For example, the hydrocolloid-based wound dressing 100 may have a modulus at 100% elongation of from about 3.5 N to about 5.5 N, about 3.75 N to about 5.25 N, about 4.0 N to about 5 N, about 4.25 N to about 4.75 N, about 4.4 N to about 4.5 N, or about 4.42 N. In another example, the hydrocolloid-based wound dressing 100 may have a modulus at 100% elongation of from about 3.5 N, about 3.8 N, about 4.0 N, about 4.2 N, or about 4.4 N to about 4.5 N, about 4.6 N, about 4.8 N, about 5.0 N, about 5.2 N, about 5.4 N, or about 5.5 N. The modulus may be evaluated according to conventional means. For example, the modulus may be evaluated on or with a Mark 10 Tensile Force Tester fitted or coupled with a digital force gauge. Test clamps of the Tensile Force Tester may be set about 25 mm apart and each of the specimen/samples (e.g., the hydrocolloid-based wound dressings 100) may be fixed to the clamps. The hydrocolloid-based wound dressings 100 may be evaluated in isolation without the carrier substrate 108, the hydrocolloid adhesive 104, and/or the release liner 106. Each of the hydrocolloid-based wound dressings 100 may be evaluated without the release liner 106 and the carrier substrate 108 (i.e., only the carrier layer 110 and the adhesive layer 104). The modulus is the force at 100% elongation of the respective specimen, and is reported in Newtons.

The backing layer 102 or the carrier layer 110 thereof (e.g., the backing layer 102 without the release liner 106), may have a modulus at 100% elongation of from about 4 Newtons (N) to about 7 N or greater. For example, the hydrocolloid-based wound dressing 100 without the release liner 106 may have a modulus at 100% elongation of from about 4 N, about 4.5N, about 5 N, or about 5.5 N to about 6 N, about 6.5 N, about 7 N, or greater. In another example, the hydrocolloid-based wound dressing 100, without the release liner 106, may have a modulus at 100% elongation of from about 4 N to about 7 N, about 4.25 N to about 6.75 N, about 4.5 N to about 6.5 N, about 4.25 N to about 6.25 N, about 4.5 N to about 6 N, about 4.75 N to about 5.75 N, or about 5.6 N.

The hydrocolloid-based wound dressing 100 may have an elongation at break of from about 550% to about 700%. For example, the hydrocolloid-based wound dressing 100 may have an elongation at break of from about 550% to about 700%, about 575% to about 675%, about 600% to about 650%, about 620% to about 640%, or about 630%. In another example, the hydrocolloid-based wound dressing 100 may have an elongation at break of from about 550%, about 575%, about 600%, about 620%, or about 625% to about 630%, about 640%, about 650%, about 675%, or about 700%. The elongation at break may be evaluated according to conventional means. For example, the elongation at break may be evaluated with a Mark 10 Tensile Force Tester fitted or coupled with a digital force gauge.

The backing layer 102 or the carrier layer 110 thereof may have an elongation at break, reported as a percentage (%) of from about 400% to about 850%. For example, the elongation at break may be from about 400% to about 850%, about 460% to about 794%, about 475% to about 779%, about 490% to about 764%, about 505% to about 749%, about 520% to about 734%, about 535% to about 719%, about 550% to about 704%, about 565% to about 689%, about 580% to about 674%, about 595% to about 659%, about 610% to about 644%, about 625% to about 629%, or about 590. In another example, the backing layer 102 or the carrier layer 110 thereof may have an elongation at break of from about 400%, about 450%, about 500%, about 550%, or about 580% to about 600%, about 650%, about 700%, about 750%, about 800%, or about 850%.

The hydrocolloid-based wound dressing 100, including the backing layer 102 and the adhesive layer 104 may have a release force from the release liner 106 or another substrate of from about 10 N to about 16 N. For example, the release force between the release liner 106 or another substrate and the adhesive layer 104 may be from about 10 N to about 16 N, about 11 N to about 15 N, about 12 N to about 14 N, or about 13 N. In another example, the release force between the release liner 106 or another substrate and the adhesive layer 104 may be from about 10 N, about 11 N, or about 12 N to about 14 N, about 15 N, or about 16 N. The release force between the release liner 106 or another substrate and the adhesive layer 104 may be conducted at an angle of about 180° C. on a tensile tester using a Mark 10 digital force gauge.

The release force between the carrier substrate 108 and the carrier layer 110 of the backing layer 102 may be from greater than 0 N to about 1 N. For example, the release force between the carrier substrate 108 and the carrier layer 110 of the backing layer 102 may be from greater than 0 N, about 0.1 N, about 0.2 N, about 0.3N, about 0.35 N, or about 0.4 N to about 0.42 N, about 0.45 N, about 0.5 N, about 0.6 N, about 0.7 N, about 0.8 N, about 0.9 N, or about 1 N. In another example, the release force between the carrier substrate 108 and the carrier layer 110 of the backing layer 102 may be from greater than 0 N to about 1 N, about 0.1 N to about 0.9 N, about 0.2 N to about 0.8 N, about 0.3 N to about 0.7 N, about 0.35 N to about 0.6 N, about 0.4 N to about 0.45 N, or about 0.41 N.

Methods for fabricating the hydrocolloid-based wound dressing 100 are described. The method may include fabricating the backing layer 102. The method may also include adhering, coupling, or otherwise contacting the backing layer 102 with the adhesive layer 104. The method may also include adhering, coupling, or otherwise contacting the release liner 106 with the adhesive layer 104.

Fabricating the backing layer 102 may include preparing the carrier film composition. Preparing the carrier film composition may include mixing, combining, or otherwise contacting one or more of the thermoplastics, the surface active agent, the additive(s), or any combination thereof with one another. For example, preparing the carrier film composition may include mixing, combining, or otherwise contacting a first polyester-based thermoplastic polyurethane elastomer, a second polyester-based thermoplastic polyurethane elastomer, a polydimethylsiloxane dispersion in a thermoplastic polyurethane, diatomaceous earth, or any combination thereof with one another to prepare the carrier film composition. The carrier film composition may be a dry composition. For example, the carrier film composition may be free or substantially free of water and/or solvents. In at least one implementation, the carrier film composition may be free or substantially free of organic solvents. For example, no organic solvents may be added during the preparation of the carrier film composition. In at least one implementation, the method may include drying (e.g., in a dryer) the carrier film composition to remove at least 70%, at least 75%, at least 80%, or more of any one or more solvents prior to fabricating the carrier layer 110.

The carrier film composition may be cast to prepare the carrier layer 110. In one example, the carrier film composition may be cast directly onto the carrier substrate 108. The carrier substrate 108 may be subsequently removed to prepare the backing layer 102 without the carrier substate 108 (e.g., only the carrier layer 110). The carrier film composition may be cast via extrusion casting to prepare the carrier layer 110. Extrusion casting or a cast extrusion process may include feeding the carrier film composition in the form of a dry composition into the cast extruder, melting and homogenizing the carrier film composition in the extruder (e.g., via a screw extruder), extruding the melted carrier film composition through a die to form the carrier layer 110 in the form of a flat film, sheet, or layer. It should be appreciated that the ability to utilize extrusion casting with the carrier film composition described herein significantly reduces the cost of preparing the backing layer 102. For example, the backing layer 102 and the carrier layer 110 thereof may be prepared without a solution casting process, which is both cost effective and environmentally friendly. As noted above, the carrier layer 110 may be extruded on the carrier substrate 108 to prepare the backing layer 102. The backing layer 102, including the carrier layer 110 and the carrier substrate 108, may be annealed. Annealing may include exposing or maintaining the backing layer 102 at a temperature of from about 35° C. to about 65° C., about 40° C. to about 60° C., or about 50° C. The backing layer 102 may be annealed for at least 5 days, at least 7 days, at least 10 days, or more.

The following numbered paragraphs are directed to one or more exemplary variations of the subject matter of the application:

1. A backing layer for a hydrocolloid-based wound dressing, comprising: a carrier film, the carrier film comprising a carrier film composition comprising: a surface active agent; and a combination of a first aromatic polyester-based thermoplastic polyurethane and a second aromatic polyester-based thermoplastic polyurethane, wherein the combination of the first aromatic polyester-based thermoplastic polyurethane and the second aromatic polyester-based thermoplastic polyurethane is configured to provide a wrinkle-free carrier film under accelerated aging conditions, and wherein the surface active agent facilitates the release of the carrier film from a carrier substrate.

2. The backing layer of paragraph 1, wherein the backing layer further comprises a carrier substrate, wherein the carrier film is disposed on the carrier substrate.

3. The backing layer of paragraph 1 or 2, wherein the weight ratio of the first aromatic polyester-based thermoplastic polyurethane to the second aromatic polyester-based thermoplastic polyurethane is from about 1.5:1 to about 2.6:1.

4. The backing layer of any one of paragraphs 1 to 3, wherein the first aromatic polyester-based thermoplastic polyurethane is present in an amount of from about 45 wt % to about 80 wt %, based on the total weight of the carrier film composition.

5. The backing layer of any one of paragraphs 1 to 4, wherein the second aromatic polyester-based thermoplastic polyurethane is present in an amount of from about 20 wt % to about 40 wt %, based on the total weight of the carrier film composition.

6. The backing layer of any one of paragraphs 1 to 5, wherein the first aromatic polyester-based thermoplastic polyurethane has a Shore Hardness, as determined according to reference test ASTM D2240, of from about 80 A to about 70 D.

7. The backing layer of any one of paragraphs 1 to 6, wherein the first aromatic polyester-based thermoplastic polyurethane has a specific gravity, as determined according to reference test ASTM D792, of from about 0.9 to about 1.5.

8. The backing layer of any one of paragraphs 1 to 7, wherein the first aromatic polyester-based thermoplastic polyurethane has a tensile strength, as determined according to reference test ASTM D412/D638, of from about 40 MPa to about 70 MPa.

9.The backing layer of any one of paragraphs 1 to 8, wherein the first aromatic polyester-based thermoplastic polyurethane has a tensile stress at about 50% elongation, according to reference test ASTM D412/D638, of from about 6 MPa to about 12 MPa.

10. The backing layer of any one of paragraphs 1 to 9, wherein the first aromatic polyester-based thermoplastic polyurethane has a tensile stress at about 100% elongation, according to reference test ASTM D412/D638, of from about 10 MPa to about 14 MPa.

11. The backing layer of any one of paragraphs 1 to 10, wherein the first aromatic polyester-based thermoplastic polyurethane has an elongation at break, according to reference test ISO 527-3/5/500 or reference test ASTM D412/D638, of from about 260% to about 320%.

12. The backing layer of any one of paragraphs 1 to 11, wherein the first aromatic polyester-based thermoplastic polyurethane has a tear strength, according to reference test ISO 34-1B, of from about 65 kN/m to about 85 kN/m.

13. The backing layer of any one of paragraphs 1 to 12, wherein the first aromatic polyester-based thermoplastic polyurethane has a Kofler Melting Point of from about 65° C. to about 95° C.

14. The backing layer of any one of paragraphs 1 to 13, wherein the first aromatic polyester-based thermoplastic polyurethane has a viscosity at about 23° C. of from about 100 centipoise (cP) to about 200 cP.

15. The backing layer of any one of paragraphs 1 to 14, wherein the first aromatic polyester-based thermoplastic polyurethane has: a peel strength adhesion to polyethylene terephthalate (PET), according to reference test TP-141, of from about 0.19 kN/m to about 0.25 kN/m; a peel strength to aluminum foil, according to reference test ASTM D1867-72, of from about 0.8 kN/m to about 1.0 kN/m; and/or a peel strength to mylar film, according to reference test ASTM D1867-72, of from about 4 kN/m to about 6 kN/m.

16. The backing layer of any one of paragraphs 1 to 15, wherein the first aromatic polyester-based thermoplastic polyurethane has a Vicat Softening Point, according to reference test ASTM D1525, of from about 40° C. to about 70° C.

17. The backing layer of any one of paragraphs 1 to 16, wherein the first aromatic polyester-based thermoplastic polyurethane has a glass transition temperature (Tg), as determined with a differential scanning calorimeter (DSC), of from about 15° C. to about 30° C.

18. The backing layer of any one of paragraphs 1 to 17, wherein the second aromatic polyester-based thermoplastic polyurethane has a Shore Hardness, as determined according to reference test ASTM D2240, of from about 70 A to about 100 A.

19. The backing layer of any one of paragraphs 1 to 18, wherein the second aromatic polyester-based thermoplastic polyurethane has a specific gravity, as determined according to reference test ASTM D792, of from about 0.9 to about 1.5.

20. The backing layer of any one of paragraphs 1 to 19, wherein the second aromatic polyester-based thermoplastic polyurethane has a tensile strength, as determined according to reference test ASTM D412/D638, of from about 30 MPa to about 70 MPa.

21. The backing layer of any one of paragraphs 1 to 20, wherein the second aromatic polyester-based thermoplastic polyurethane has an elongation at break, according to reference test ISO 527-3/5/500 or reference test ASTM D412/D638, of from about 350% to about 600%.

22. The backing layer of any one of paragraphs 1 to 21, wherein the second aromatic polyester-based thermoplastic polyurethane has a 100% modulus at 100% strain, according to reference test ASTM D412/D638 of the ASTM, of from about 3.0 MPa to about 7 MPa.

23. The backing layer of any one of paragraphs 1 to 22, wherein the second aromatic polyester-based thermoplastic polyurethane has a 300% modulus at 300% strain, according to reference test ASTM D412/D638 of the ASTM, of from about 10 MPa to about 16 MPa.

24. The backing layer of any one of paragraphs 1 to 23, wherein the second aromatic polyester-based thermoplastic polyurethane has a melt viscosity at about 204° C., according to reference test ASTM D1084/88, of from about 24,000 cP to about 32,000 cP.

25. The backing layer of any one of paragraphs 1 to 24, wherein the second aromatic polyester-based thermoplastic polyurethane has: a peel strength adhesion to polyethylene terephthalate (PET), according to reference test TP-141, of from about 0.09 kN/m to about 0.13 kN/m; a peel strength to aluminum foil, according to reference test ASTM D1867-72, of from about 0.8 kN/m to about 1.0 kN/m; and/or a peel strength to mylar film, according to reference test ASTM D1867-72, of from about 4 kN/m to about 6 kN/m.

26. The backing layer of any one of paragraphs 1 to 25, wherein the second aromatic polyester-based thermoplastic polyurethane has a glass transition temperature (Tg), as determined with a differential scanning calorimeter (DSC), of from about 15° C. to about 30° C.

27. The backing layer of any one of paragraphs 1 to 26, wherein the second aromatic polyester-based thermoplastic polyurethane has a ring and ball softening point, as determined according to ASTM E2892, of from about 150° C. to about 170° C.

28. The backing layer of any one of paragraphs 1 to 27, wherein the surface active agent comprises one or more of a silicone, a siloxane, polytetrafluoroethylene (PTFE), an antistatic agent, or a combination thereof.

29. The backing layer of any one of paragraphs 1 to 28, wherein the surface active agent comprises the siloxane dispersed in an organic resin.

30. The backing layer of any one of paragraphs 28 to 29, wherein the siloxane comprises a polydimethylsiloxane, and wherein the organic resin comprises a thermoplastic polyurethane resin.

31. The backing layer of any one of paragraphs 28 to 30, wherein the siloxane is present in an amount of from greater than 0 wt % to about 8 wt %, based on the total weight of the carrier film composition.

32. The backing layer of any one of paragraphs 1 to 31, wherein the carrier film composition further comprises an additive.

33. The backing layer of paragraph 32, wherein the additive comprises diatomaceous earth.

34. The backing layer of paragraph 33, wherein the diatomaceous earth has an average particle diameter of from about 1 μm to about 10 μm.

35. The backing layer of any one of paragraphs 1 to 34, wherein the backing layer has a moisture vapor transmission rate, according to reference test ASTM E96-00, of from about 450 g/m²/day to about 600 g/m²/day.

36. The backing layer of any one of paragraphs 1 to 35, wherein the backing layer has a modulus at 100% elongation of from about 4 Newtons (N) to about 7 N.

37. The backing layer of any one of paragraphs 1 to 36, wherein the backing layer has an elongation at break of from about 400% to about 850%.

38. The backing layer of any one of paragraphs 2 to 37, wherein a release force between the carrier film and the carrier substrate is from greater than 0 N to about 1 N.

39. A hydrocolloid-based wound dressing, comprising: the backing layer of any one of paragraphs 1 to 38; and an adhesive layer disposed on the backing layer.

40. The hydrocolloid-based wound dressing of paragraph 39, wherein the adhesive layer comprises a hydrocolloid and a tackifier.

41. The hydrocolloid-based wound dressing of paragraph 39 or 40, further comprising a release liner disposed on the adhesive layer.

42. The hydrocolloid-based wound dressing of any one of paragraphs 39 to 41, wherein the hydrocolloid-based wound dressing has a moisture vapor transmission rate, according to reference test ASTM E96-00, of from about 100 g/m²/day to about 180 g/m²/day.

43. The hydrocolloid-based wound dressing of any one of paragraphs 39 to 42, wherein the hydrocolloid-based wound dressing has a modulus at 100% elongation of from about 3.5 N to about 5.5 N.

44. The hydrocolloid-based wound dressing of any one of paragraphs 39 to 43, wherein the hydrocolloid-based wound dressing has an elongation at break of from about 550% to about 700%.

45. The hydrocolloid-based wound dressing of any one of paragraphs 39 to 44, wherein a release force between the release liner and the adhesive layer is from about 10 N to about 16 N.

46. A method for preparing the backing layer of any one of paragraphs 2 to 38, the method comprising: contacting the first aromatic polyester-based thermoplastic polyurethane, the second aromatic polyester-based thermoplastic polyurethane, and the surface active agent with one another to prepare a mixture; melting and homogenizing the mixture in an extruder; and extrusion casting the melted mixture on the carrier substrate to prepare the backing layer.

47. The method of paragraph 46, further comprising drying the mixture prior to melting and homogenizing the mixture in the extruder.

48. The method of any one of paragraphs 46 and 47, wherein the mixture is substantially free of organic solvents.

49. The method of any one of paragraphs 46 to 48, further comprising annealing the backing layer after extrusion casting the melted mixture on the carrier substrate.

### EXAMPLES

The examples and other implementations described herein are exemplary and not intended to be limiting in describing the full scope of compositions and methods described herein. Equivalent changes, modifications, and variations of specific implementations, materials, compositions, and methods may be made within the scope of the implementations or embodiments described herein, with substantially similar results.

### Example 1

Exemplary carrier film compositions (1)-(8) were prepared and evaluated with comparative or conventional carrier film compositions (C1) and (C2). Specifically, exemplary carrier film compositions (1)-(8) were prepared by combining the components/ingredients according to Table 1. The components of the carrier film compositions (1)-(8) were dry blended and subsequently dried overnight. Each of the dry blends of the respective carrier film compositions (1)-(8) was then melt extrusion cast on a carrier substrate to prepare respective backing layers including the carrier substrate and the carrier film. The carrier substrate was a paper film carrier having a thickness of about 3.6 mm, which was commercially available from Ahlstrom-Munksjö of Helsinki, Finland. The carrier substrate had a release coating on both sides thereof. Particularly, a first side of the carrier substrate was coated with an ethylene acrylic acid emulsion and a second side of the carrier substrate was coated with a silicone release coating. The melt extrusion casting was conducted on a single screw extruder with a barrier screw and a L/D ratio of about 40 to about 1 (about 40:1). After melt extrusion casting, each of the resulting backing layers were annealed at about 50° C. for about 7 days. The exemplary backing layers prepared from melt extrusion casting each of the carrier film compositions (1)-(8) on the carrier substrate had an overall thickness of about 0.25 mm. Specifically, the respective extruded carrier layer prepared from each of the carrier film compositions (1)-(8) had a thickness of about 0.1 mm, and the carrier substrate (e.g., paper film carrier) had a thickness of about 0.15 mm. The comparative carrier film compositions (C1) and (C2) were similarly utilized to prepare comparative backing layers. It is noted that the specific components of the comparative carrier film compositions (C1) and (C2) were not known; however, the comparative carrier film composition (C1) was an aromatic polyester type thermoplastic polyurethane (TPU) based composition, and the comparative carrier film composition (C2) was an aromatic polyether type TPU based composition. Further, the comparative backing layer prepared from the comparative carrier film composition (C1) was solution cast, and the comparative backing layer prepared from the comparative carrier film composition (C2) was melt extrusion cast.

TABLE 1

| | CARRIER FILM COMPOSITION | | | | | | | |
|---|---|---|---|---|---|---|---|---|
| COMPONENT | (1) | (2) | (3) | (4) | (5) | (6) | (7) | (8) |
| Aromatic Polyester Type TPU | 38 | 52 | 67 | 57 | 52 | 57 | 52 | 48 |
| Shore A 90¹ (wt %) | | | | | | | | |
| Aromatic Polyester Type TPU | 58 | 44 | 29 | 30 | 28 | 30 | 28 | 26 |
| Shore A 85² (wt %) | | | | | | | | |
| Silicone Polymer in TPU³ | 4 | 4 | 4 | 4 | 4 | 4 | 4 | 4 |
| (wt %) | | | | | | | | |
| Diatomaceous Earth in TPU⁴ | — | — | — | 9 | — | — | — | — |
| (wt %) | | | | | | | | |
| Diatomaceous Earth in TPU⁵ | — | — | — | — | 16 | — | — | — |
| (wt %) | | | | | | | | |
| Diatomaceous Earth in TPU⁶ | — | — | — | — | — | 9 | 16 | 22 |
| (wt %) | | | | | | | | |

¹Pearlstick TPU 5715 ™;
²Pearlstick TPU 5701 ™;
³50% Silicone polymer in aromatic polyester TPU carrier;
⁴40% Superfine diatomaceous earth (DE) powder (about 5 μm particles) and 0.3% ethylene bis(stearamide) (EBS) wax in Thermoplastic Polyether-Polyurethane TPU carrier, BASF Elastollan ® 1180;
⁵40% Superfine diatomaceous earth (DE) powder (about 5 μm particles) and about 0.3% fumed silica Aerosol ® 200 dusted pellets in aromatic polyester type TPU carrier, Pearlstick ™ TPU 5715;
⁶40% Superfine diatomaceous earth (DE) powder (about 5 μm particles) in Pearlstick ™ TPU 5701 carrier.

Each of the backing layers, and the respective carrier films thereof, prepared from the exemplary carrier film compositions (1)-(8) or the comparative carrier film compositions (C1) and (C2) were utilized to prepare hydrocolloid-based wound dressings or laminates. The dressings or laminates were prepared by disposing a layer of a hydrocolloid adhesive on the respective carrier film of the backing layer and disposing a film release liner on the respective hydrocolloid adhesive, thereby preparing the hydrocolloid-based wound dressing as exemplified in FIG. 1. Specifically, about 20 g of the hydrocolloid adhesive composition was pressed between a silicone-coated paper film release liner (thickness of about 170 μm) and each of the backing layers prepared from the exemplary carrier film compositions (1)-(8) or the comparative carrier film compositions (C1) and (C2). The respective hydrocolloid adhesive layer of each of the resulting laminates had a thickness of about 0.7 mm-1.0 mm.

### Example 2

The efficacy of each of the hydrocolloid-based wound dressings and the respective carrier layer thereof, prepared from the exemplary carrier film compositions (1)-(8) or the comparative carrier film compositions (C1) and (C2), were evaluated. Specifically, the hydrocolloid-based wound dressings and the respective carrier layer thereof were evaluated for one or more of the following properties: (1) modulus at 100% elongation; (2) elongation at break; (3) moisture vapor transmission rate (MVTR); (4) thickness; (5) film release liner removal; and (6) visual properties (e.g., wrinkling, delamination, etc.). It should be noted that some of the properties, such as the modulus at 100% elongation, elongation at break, and MVTR, of each of the hydrocolloid-based wound dressings were evaluated without the film release liner, as further specified below. Similarly, some of the properties of the carrier layer were evaluated without the carrier substrate, as further specified below.

Moisture vapor transmission rate (MVTR) was evaluated according to reference test ASTM E96-00 of the American Society for Testing and Materials (ASTM). Specifically, the MVTR test method utilized MVTR measurement cups. The cup was upside down, or inverted, and filled with liquid (0.9% saline water solution) so that the liquid touches the PU film. The temperature of the testing was at 37° C./24 hours using the following: steel Cup, 9 ml saline water, 2″ diameter PU film, rubber steel lid ring, and then clamped in the oven at 37° C. The RH of the testing environment was about 30%. In the present examples, MVTR was evaluated for the carrier film without the carrier substrate or with the carrier substrate removed. For example, the carrier film prepared from each of the carrier film compositions (1)-(8) and the comparative carrier film compositions (C2) and (C3) were evaluated for their respective MVTR. The MVTR of each of the hydrocolloid-based wound dressings was also evaluated without the film release liner. The results are summarized in Tables 2 and 3.

The thickness was evaluated according to conventional methods. For example, the thickness of each of the backing layers, including the carrier substrate and the carrier film, was evaluated with a micrometer. The micrometer was capable of measuring film thicknesses of about 0.001 mm. The thickness of the hydrocolloid adhesive layer was evaluated by a subtraction method. For example, the thickness of the hydrocolloid adhesive layer was determined by subtracting the thickness of the film release liner and the backing layer, including the carrier substrate and the carrier film, from a total thickness of the hydrocolloid-based wound dressing. The results are summarized in Tables 2 and 3.

The liner release/removal test measured a release force between the film release liner and the hydrocolloid adhesive layer. To evaluate the release force, a specimen having a length and width of about 50 mm (+/−1.5 mm) and 55 mm (+/−1.5 mm), respectively, was utilized. The liner removal test was conducted on the specimen at an angle of about 180° on a tensile tester using a Mark 10 digital force gauge. Specifically, the specimen was fixed to the tensile tester by clamping the film release liner to a moveable clamp and clamping the hydrocolloid adhesive and the backing layer to a fixed clamp. The liner removal test was conducted at a clamp speed of about 300 mm/min and the force gauge measured the force in Newtons (N). Each of the hydrocolloid-based wound dressings were evaluated in plurality, and a truncated mean or trimmed mean was determined. Specifically, a 5% trimmed mean of the liner release force (N) was determined for each of the hydrocolloid-based wound dressings. The results are summarized in Tables 2 and 3.

The film release/removal test was also utilized to measure the release force between the carrier substrate and the respective layer of the carrier film compositions (1)-(8), (C1), and (C2). The film removal test was conducted as described above with respect to the liner removal test, which measured the release force between the film release liner and the hydrocolloid adhesive layer. It is noted, however, that the specimen utilized in the film removal test had a length and width of about 1,295 mm (+/−1.5 mm) and 55 mm (+/−1.5 mm), respectively. The results are summarized in Tables 2 and 3.

The modulus and elongation at break were evaluated according to conventional methods. For example, the modulus and elongation were evaluated on a Mark 10 Tensile Force Tester fitted or coupled with a digital force gauge. Test clamps of the Tensile Force Tester were set about 25 mm apart and each of the specimen were fixed to the clamps. The respective carrier film of each of the hydrocolloid-based wound dressings was evaluated in isolation without the carrier substrate, the hydrocolloid adhesive, and the release liner. Each of the hydrocolloid-based wound dressings were evaluated without the release liner and the carrier substrate (i.e., only the carrier film and the hydrocolloid adhesive). The modulus is the force at 100% elongation of the respective specimen, and is reported in Newtons. The elongation at break was evaluated in percentage and represents the extension of the specimen when the specimen is broken. The results are summarized in Tables 2 and 3.

TABLE 2
Properties of Carrier Film

| | CARRIER FILM COMPOSITION | | | | | | | | | |
|---|---|---|---|---|---|---|---|---|---|---|
| Property | (1) | (2) | (3) | (4) | (5) | (6) | (7) | (8) | (C1) | (C2) |
| Film | 0.039 | 0.023 | 0.041 | 0.032 | 0.034 | 0.031 | 0.021 | 0.033 | 0.030 | 0.024 |
| Thickness (mm) | | | | | | | | | | |
| Modulus | 4.1 | 4.0 | 6.0 | 4.8 | 5.6 | 5.1 | — | 5.2 | 3.9 | 3.7 |
| at 100% | | | | | | | | | | |
| Elongation | | | | | | | | | | |
| (N) | | | | | | | | | | |
| Elongation | 662 | 657 | 584 | 493 | 590 | 654 | — | 706 | 502 | 687 |
| at Break | | | | | | | | | | |
| (%) | | | | | | | | | | |
| Film Release | 0.64 | 1.18 | 0.92 | 0.64 | 0.41 | 0.07 | — | 0.07 | 0.56 | 0.30 |
| Force from | | | | | | | | | | |
| Carrier | | | | | | | | | | |
| Substrate | | | | | | | | | | |
| (N/25 mm) | | | | | | | | | | |
| MVTR | 543 | 580 | 892 | — | 536 | 584 | — | 509 | 551 | 1601 |
| (g/m²/day) at | | | | | | | | | | |
| 37° C. | | | | | | | | | | |

TABLE 3
Properties of Hydrocolloid-Based Wound Dressing

| | CARRIER FILM COMPOSITION | | | | | | | | | |
|---|---|---|---|---|---|---|---|---|---|---|
| | UTILIZED IN DRESSING | | | | | | | | | |
| Property | (1) | (2) | (3) | (4) | (5) | (6) | (7) | (8) | (C1) | (C2) |
| Adhesive | — | — | 0.50 | — | 0.49 | — | — | — | 0.44 | 0.41 |
| Thickness (mm) | | | | | | | | | | |
| MVTR | — | — | 121 | — | 139 | — | — | — | 113 | 201 |
| (g/m²/day) | | | | | | | | | | |
| at 37° C. | | | | | | | | | | |
| Peel Adhesion | — | — | 11.1 | — | 13.0 | — | — | — | 7.80 | 9.66 |
| (N/25 mm) | | | | | | | | | | |
| Elongation | — | — | 128 | — | 627 | — | — | — | 571 | 814 |
| at Break (%) | | | | | | | | | | |
| Modulus | — | — | 5.1 | — | 4.42 | — | — | — | 3.10 | 6.99 |
| (N/24 mm) | | | | | | | | | | |

### Example 3

Each of the hydrocolloid-based wound dressings prepared in Example 1 were subjected to accelerated aging conditions to determine the performance and/or stability for a simulated storage/service life of at least 5 years. Specifically, each of the hydrocolloid-based wound dressings were maintained at about 40° C. and 30% relative humidity (RH) for about 7 days. The physical properties of each of the hydrocolloid-based wound dressings were observed before and after exposing the hydrocolloid-based wound dressings to the accelerated aging conditions, and a pass/fail was determined based, at least, on film shrinkage or wrinkling of the hydrocolloid-based wound dressings or components thereof. The results are summarized in Table 4.

TABLE 4
Properties of Aged Hydrocolloid-Based Wound Dressing/Laminate

| Wound | | |
|---|---|---|
| Dressing/Laminate | Pass/Fail | Physical Observation |
| (1) | Fail | Film Shrinkage |
| (2) | Fail | Film Shrinkage |
| (3) | Pass | No Film Shrinkage |
| (4) | Fail | Film Shrinkage |
| (5) | Pass | No Film Shrinkage |
| (6) | Fail | Film Shrinkage |
| (7) | Fail | Film Shrinkage |
| (8) | Fail | Film Shrinkage |
| (C1) | Pass | No Film Shrinkage |
| (C2) | Fail | Film Shrinkage |

As indicated in Table 4, the hydrocolloid-based wound dressing utilizing the carrier film compositions (3) and (5) demonstrated relatively greater stability and efficacy after accelerated aging conditions as compared to the remaining hydrocolloid-based wound dressings as no film shrinkage or wrinkling thereof was observed. It is further noted that one or more of the properties (e.g., MVTR, elongation, modulus, film release, etc.) of the hydrocolloid-based wound dressings prepared from the carrier film compositions (3) and (5) exhibited comparable or enhanced properties as compared to the hydrocolloid-based wound dressing prepared from the conventional carrier film compositions (C1) and (C2).

While the devices, systems, and methods have been described in detail herein in accordance with certain preferred implementations thereof, many modifications and changes therein may be affected by those skilled in the art. Accordingly, the foregoing description should not be construed to be limited thereby but should be construed to include such aforementioned obvious variations and be limited only by the spirit and scope of the following claims.

What is claimed is:

1. A backing layer for a hydrocolloid-based wound dressing, comprising:
a carrier film, the carrier film comprising a carrier film composition comprising:
a surface active agent; and
a combination of a first aromatic polyester-based thermoplastic polyurethane and a second aromatic polyester-based thermoplastic polyurethane,
wherein the combination of the first aromatic polyester-based thermoplastic polyurethane and the second aromatic polyester-based thermoplastic polyurethane is configured to provide a wrinkle-free carrier film under accelerated aging conditions, and
wherein the surface active agent facilitates the release of the carrier film from a carrier substrate.

2. The backing layer of claim 1, wherein the backing layer further comprises a carrier substrate, wherein the carrier film is disposed on the carrier substrate.

3. The backing layer of claim 1, wherein the weight ratio of the first aromatic polyester-based thermoplastic polyurethane to the second aromatic polyester-based thermoplastic polyurethane is from about 1.5:1 to about 2.6:1.

4. The backing layer of claim 1, wherein the first aromatic polyester-based thermoplastic polyurethane is present in an amount of from about 45 wt % to about 80 wt %, based on the total weight of the carrier film composition.

5. The backing layer of claim 1, wherein the second aromatic polyester-based thermoplastic polyurethane is present in an amount of from about 20 wt % to about 40 wt %, based on the total weight of the carrier film composition.

6. The backing layer of claim 1, wherein the first aromatic polyester-based thermoplastic polyurethane has a Shore Hardness, as determined according to reference test ASTM D2240, of from about 80 A to about 70 D.

7. The backing layer of claim 1, wherein the first aromatic polyester-based thermoplastic polyurethane has a tensile strength, as determined according to reference test ASTM D412/D638, of from about 40 MPa to about 70 MPa.

8. The backing layer of claim 1, wherein the first aromatic polyester-based thermoplastic polyurethane has a tensile stress at about 50% elongation, according to reference test ASTM D412/D638, of from about 6 MPa to about 12 MPa.

9. The backing layer of claim 1, wherein the first aromatic polyester-based thermoplastic polyurethane has an elongation at break, according to reference test ISO 527-3/5/500 or reference test ASTM D412/D638, of from about 260% to about 320%.

10. The backing layer of claim 1, wherein the first aromatic polyester-based thermoplastic polyurethane has a tear strength, according to reference test ISO 34-1B, of from about 65 kN/m to about 85 kN/m.

11. The backing layer of claim 1, wherein the first aromatic polyester-based thermoplastic polyurethane has a viscosity at about 23° C. of from about 100 centipoise (cP) to about 200 cP.

12. The backing layer of claim 1, wherein the second aromatic polyester-based thermoplastic polyurethane has a Shore Hardness, as determined according to reference test ASTM D2240, of from about 70 A to about 100 A.

13. The backing layer of claim 1, wherein the second aromatic polyester-based thermoplastic polyurethane has a tensile strength, as determined according to reference test ASTM D412/D638, of from about 30 MPa to about 70 MPa.

14. The backing layer of claim 1, wherein the second aromatic polyester-based thermoplastic polyurethane has an elongation at break, according to reference test ISO 527-3/5/500 or reference test ASTM D412/D638, of from about 350% to about 600%.

15. The backing layer of claim 1, wherein the second aromatic polyester-based thermoplastic polyurethane has a melt viscosity at about 204° C., according to reference test ASTM D1084/88, of from about 24,000 cP to about 32,000 cP.

16. The backing layer of claim 1, wherein the surface active agent comprises one or more of a silicone, a siloxane, polytetrafluoroethylene (PTFE), an antistatic agent, or a combination thereof.

17. The backing layer of claim 16, wherein the surface active agent comprises the siloxane dispersed in an organic resin.

18. The backing layer of claim 17, wherein the siloxane comprises a polydimethylsiloxane, and wherein the organic resin comprises a thermoplastic polyurethane resin.

19. The backing layer of claim 18, wherein the siloxane is present in an amount of from greater than 0 wt % to about 8 wt %, based on the total weight of the carrier film composition.

20. The backing layer of claim 1, wherein the carrier film composition further comprises an additive.

21. The backing layer of claim 20, wherein the additive comprises diatomaceous earth.
